(19) United States
(12) Patent Application Publication
(10) Pub. No.: US 2003/0134307 A1
(43) Pub. Date: Jul. 17, 2003

(54) Asymmetric PCR with nuclease-free polymerase or nuclease-resistant molecular beacons

(75) Inventors: Kenneth B. Beckman, Alameda, CA (US); Robert D. Larsen, Alameda, CA (US); Kathleen Y. Lee, Oakland, CA (US)

Correspondence Address: QUINE INTELLECTUAL PROPERTY LAW GROUP, P.C., P O BOX 458, ALAMEDA, CA, 94501, US

(73) Assignee: Gorilla Genomics, Inc., Alameda, CA

(21) Appl. No.: 10/281,054

(22) Filed: Oct. 25, 2002

Related U.S. Application Data
(60) Provisional application No. 60/346,263, filed on Oct. 25, 2001.
(60) Provisional application No. 60/336,851, filed on Oct. 30, 2001.

(51) Int. Cl. C12Q 1/68; C12P 19/34

(52) U.S. Cl. 435/6; 435/91.2

(57) ABSTRACT

This invention provides methods for performing combined asymmetric amplification (e.g., asymmetric PCR amplification) and detection of nucleic acid targets using molecular beacons to detect the products. Methods using a polymerase having reduced or eliminated 5′ to 3′ nuclease activity are provided, as are methods using nuclease-resistant molecular beacons. Asymmetric amplifications using nuclease-free polymerase or nuclease-resistant molecular beacons provide dramatic improvements in signal intensity detected as a result of molecular beacon binding to a target nucleic acid, e.g., during asymmetric PCR. Attendant compositions, systems, devices and kits are also features of the invention.

### CROSS-REFERENCE TO RELATED APPLICATIONS

This application is a non-provisional utility patent application claiming priority to and benefit of the following prior provisional patent applications: U.S. S No. 60/346,263 filed Oct. 25, 2001, entitled “Asymmetric PCR with Nuclease-Free Polymerase” by Robert D. Larsen and Kenneth B. Beckman, and U.S. S No. 60/336,851 filed Oct. 30, 2001, all entitled “Asymmetric PCR with Nuclease-Free Polymerase” by Robert D. Larsen and Kenneth B. Beckman. The present application claims priority to and benefit of each of these prior applications, each of which is incorporated by reference.

### FIELD OF THE INVENTION

The present invention is in the field of molecular beacons and PCR, particularly asymmetric PCR.

### BACKGROUND OF THE INVENTION

Molecular beacons (MBs) are oligonucleotides, which can be comprised of standard or modified nucleotides or analogs thereof (e.g., peptide nucleic acids (PNAs)), designed for the detection and quantification of target nucleic acids (e.g., target DNAs). The basic principles of molecular beacon-mediated target nucleic acid detection are taught, e.g., in co-pending application U.S. S No. PCT/US01/13719.

As taught in the '719 application, the 5′ and 3′ termini of the MB collectively comprise a pair of moieties which confers the detectable properties of the MB. Typically, one of the termini is attached to a fluorophore and the other to a quencher molecule capable of quenching a fluorescent emission of the fluorophore. For example, one example fluorophore-quencher pair can use a fluorophore such as EDANS or fluorescein, e.g., on the 5′-end, and a quencher such as Dabcyl, e.g., on the 3′-end.

When the MB is present free in solution, i.e., not hybridized to a second nucleic acid, the stem of the MB is stabilized by complementary base pairing. This self-complementary pairing results in a “stem-loop” (also called a “hairpin” or “hairpin loop”) structure for the MB in which the fluorescent and the quenching moieties are proximal to one another. In this conformation, the fluorophore is quenched by the quencher.

The loop of the molecular beacon is complementary to a sequence to be detected in the target nucleic acid, such that hybridization of the loop to its complementary sequence in the target forces disassociation of the stem, thereby distancing the fluorophore and quencher from each other. This results in unquenching of the fluorophore, causing an increase in fluorescence of the MB.

Further details regarding standard methods of making and using MBs are well established in the literature, and MBs are available from a number of commercial reagent sources. Further details regarding methods of MB manufacture and use are found, e.g., in Leone et al. (1995) “Molecular beacon probes combined with amplification by NASBA enable homogenous real-time detection of RNA.” Nucleic Acids Res. 26:2150-2155; Tyagi and Kramer (1996) “Molecular beacons: probes that fluoresce upon hybridization” Nature Biotechnology 14:303-308; Blok and Kramer (1997) “Amplifiable hybridization probes containing a molecular switch” Mol Cell Probes 11:187-194; Hsuih et al. (1997) “Novel, ligation-dependent PCR assay for detection of hepatitis C in serum” J Clin Microbiol 34:501-507; Kostrikis et al. (1998) “Molecular beacons: spectral genotyping of human alleles” Science 279:1228-1229; Sokol et al. (1998) “Real time detection of DNA:RNA hybridization in living cells” Proc. Natl. Acad. Sci. U.S.A. 95:11538-11543; Tyagi et al. (1998) “Multicolor molecular beacons for allele discrimination” Nature Biotechnology 16:49-53; Bonnet et al. (1999) “Thermodynamic basis of the chemical specificity of structured DNA probes” Proc. Natl. Acad. Sci. U.S.A. 96:6171-6176; Fang et al. (1999) “Designing a novel molecular beacon for surface-immobilized DNA hybridization studies” J. Am. Chem. Soc. 121:2921-2922; Marras et al. (1999) “Multiplex detection of single-nucleotide variation using molecular beacons” Genet. Anal. Biomol. Eng. 14:151-156; and Vet et al. (1999) “Multiplex detection of four pathogenic retroviruses using molecular beacons” Proc. Natl. Acad. Sci. U.S.A. 96:6394-6399. Additional details regarding MB construction and use are found in the patent literature, e.g., U.S. Pat. No. 5,925,517 (Jul. 20, 1999) to Tyagi et al. entitled “Detectably labeled dual conformation oligonucleotide probes, assays and kits;” U.S. Pat. No. 6,150,097 to Tyagi et al (Nov. 21, 2000) entitled “Nucleic acid detection probes having non-FRET fluorescence quenching and kits and assays including such probes” and U.S. Pat. No. 6,037,130 to Tyagi et al (Mar. 14, 2000), entitled “Wavelength-shifting probes and primers and their use in assays and kits.”

MBs are gaining wide spread acceptance as robust reagents for detecting and quantitating nucleic acids, including in real time (e.g., MBs can be used to detect targets as they are formed, e.g., by PCR). A variety of commercial suppliers produce standard and custom molecular beacons, including Cruachem (cruachem.com), Oswel Research Products Ltd. (UK; oswel.com), Research Genetics (a division of Invitrogen, Huntsville Ala. (resgen.com)), the Midland Certified Reagent Company (Midland, Tex. mcrc.com) and Gorilla Genomics, Inc. (Alameda, Calif.).

Despite such widespread acceptance and commercial development of MBs and related technologies, there remain a number of areas for improvement in the design and use of MBs. The present invention provides new asymmetric PCR strategies using MBs with nuclease-free DNA polymerases or using nuclease resistant MBs. These strategies greatly improve the signal intensity, sensitivity, and quantitative nature of MB detection strategies, e.g., for real time PCR product detection.

### SUMMARY OF THE INVENTION

The present invention provides methods in which MBs are used in conjunction with asymmetric amplification (e.g., asymmetric PCR amplification) for detection of a nucleic acid target. In one class of embodiments, the enzyme used for the amplification (e.g., a DNA polymerase) has reduced or eliminated (e.g., undetectable) 5′-3′ nuclease activity. In another class of embodiments, the MBs are nuclease-resistant. Compositions, systems, devices and kits that relate to each of the methods are also a feature of the invention.

Thus, in a first general class of embodiments, the invention provides new asymmetric amplification strategies (e.g., asymmetric PCR strategies) using nuclease-free polymerase to enhance MB-mediated detection of a nucleic acid target. In the methods, a molecular beacon, a first primer, a second primer, a template nucleic acid, and a polymerase substantially lacking 5′ to 3′ nuclease activity are provided. The molecular beacon comprises a region of complementarity to a first region of a first strand of a nucleic acid target. The first primer comprises a region of identity with a second region of the first strand of the nucleic acid target, and the second primer comprises a region of complementarity to a third region of the first strand of the nucleic acid target. The third region is 3′ of the first region, and the first region is 3′ of the second region, such that the two primers flank the nucleic acid target. The first primer is provided at a concentration that is at least about 1.3 times (e.g., at least about two times, at least about three times, or more) that of the second primer. The template nucleic acid comprises the first strand of the nucleic acid target, a second strand of the nucleic acid target that is complementary to the first strand, or both. The target nucleic acid is amplified by subjecting the template nucleic acid, the first and second primers, the molecular beacon, and the polymerase (e.g., a thermostable DNA polymerase) to cycles (e.g., thermal cycles) comprising denaturation, annealing, and extension steps. A signal (e.g., a fluorescent emission) from the molecular beacon is detected at at least one time point during or after the cycles (e.g., at least once during each annealing step). The methods can be applied to various forms of PCR, including, but not limited, to real-time quantitative PCR, reverse transcription PCR (rt-PCR), in situ PCR, and/or multiplex PCR, and can be used for single nucleotide discrimination (e.g., SNP detection, allele discrimination, and the like).

A second general class of embodiments provides new asymmetric amplification strategies (e.g., asymmetric PCR strategies) using nuclease-resistant MBs to enhance MB-mediated detection of a nucleic acid target. In the methods, a molecular beacon, a first primer, a second primer, a template nucleic acid, and a polymerase are provided. The molecular beacon comprises a region of complementarity to a first region of a first strand of a nucleic acid target, and the MB is resistant to 5′ to 3′ nuclease activity. The first primer comprises a region of identity with a second region of the first strand of the nucleic acid target, and the second primer comprises a region of complementarity to a third region of the first strand of the nucleic acid target. The third region is 3′ of the first region, and the first region is 3′ of the second region. The first primer is provided at a concentration that is at least about 1.3 times (e.g., at least about two times, at least about three times, or more) that of the second primer. The template nucleic acid comprises the first strand of the nucleic acid target, a second strand of the nucleic acid target that is complementary to the first strand, or both. The target nucleic acid is amplified by subjecting the template nucleic acid, the first and second primers, the molecular beacon, and the polymerase (e.g., a thermostable DNA polymerase) to cycles (e.g., thermal cycles) comprising denaturation, annealing, and extension steps. A signal (e.g., a fluorescent emission) from the molecular beacon is detected at at least one time point during or after the cycles (e.g., at least once during each annealing step). The nuclease-resistant MB can comprise, for example, a peptide nucleic acid, one or more 2′-O-methyl nucleotides, and/or one or more phosphorothioate linkages. The methods can be applied to various forms of PCR, including, but not limited to, real-time quantitative PCR, rt-PCR, in situ PCR, and/or multiplex PCR, and can be used for single nucleotide discrimination (e.g., SNP detection, allele discrimination, and the like).

The present invention also includes compositions, e.g., for practicing the methods herein or that are produced by the methods herein. For example, the invention provides a composition comprising a molecular beacon, a first primer, a second primer, and a polymerase substantially lacking 5′ to 3′ nuclease activity. The molecular beacon comprises a region of complementarity to a first region of a first strand of a nucleic acid target. The first primer comprises a region of identity with a second region of the first strand of the nucleic acid target, and the second primer comprising a region of complementarity to a third region of the first strand of the nucleic acid target. The third region is 3′ of the first region, and the first region is 3′ of the second region. The first primer is present at a concentration that is at least about 1.3 times (e.g., at least about two times, at least about three times, or more) that of the second primer.

Another class of embodiments provides a composition comprising a molecular beacon, a first primer, and a second primer. The molecular beacon comprises a region of complementarity to a first region of a first strand of a nucleic acid target, and the MB is resistant to 5′ to 3′ nuclease activity. The first primer comprises a region of identity with a second region of the first strand of the nucleic acid target, and the second primer comprising a region of complementarity to a third region of the first strand of the nucleic acid target. The third region is 3′ of the first region, and the first region is 3′ of the second region. The first primer is present at a concentration that is at least about 1.3 times (e.g., at least about two times, at least about three times, or more) that of the second primer.

Kits, e.g., comprising components of the compositions, e.g., in conjunction with packaging materials, containers, and/or instructions for use of the compositions of the invention, e.g., in conjunction with the methods of the invention, provide another class of embodiments of the invention.

### BRIEF DESCRIPTION OF THE DRAWINGS

FIG. 1 is an amplification plot, showing the fluorescence measured at each cycle, for symmetric and asymmetric PCR amplification of cDNA target F6 using a nuclease-free polymerase.

FIG. 2 is an amplification plot, showing the fluorescence measured at each cycle, for symmetric and asymmetric PCR amplification of cDNA target E2 using a nuclease-free polymerase.

FIG. 3 is an amplification plot, showing the fluorescence measured at each cycle, for symmetric and asymmetric PCR amplification of cDNA target E5 using a nuclease-free polymerase.

FIG. 4 is an amplification plot, showing the fluorescence measured at each cycle, for symmetric and asymmetric PCR amplification of cDNA target A2 using a nuclease-free polymerase.

FIG. 5 is an amplification plot, showing the fluorescence measured at each cycle, for symmetric and asymmetric PCR amplification of cDNA target B1 using a nuclease-free polymerase.

FIG. 6 is an amplification plot, showing the fluorescence measured at each cycle, for symmetric and asymmetric PCR amplification of cDNA target A5 using a nuclease-free polymerase.

FIG. 7 is an amplification plot, showing the fluorescence measured at each cycle, for symmetric and asymmetric PCR amplification of cDNA target B2 using a nuclease-free polymerase.

FIG. 8 is an amplification plot, showing the fluorescence measured at each cycle, for symmetric and asymmetric PCR amplification of cDNA target A6 using a nuclease-free polymerase.

FIG. 9, Panels A-D schematically depict an asymmetric PCR amplification using nuclease-free polymerase and a molecular beacon.

FIG. 10, Panels A-D schematically depict an asymmetric PCR amplification with a nuclease-resistant molecular beacon.

### DEFINITIONS

The following definitions supplement those in the art and are directed to the current application and are not to be imputed to any related or unrelated case, e.g., to any commonly owned patent or application.

“Fixed cells” are cells that have been treated (e.g., chemically treated) to strengthen cellular structures (e.g., membranes) against disruption (e.g., by temperature changes, solvent changes, mechanical stress or drying). Cells can be fixed, e.g., in suspension or as part of a tissue sample. Treatment with proteinases, surfactants, organic solvents or the like can be used to modify (e.g., to increase) the permeability of fixed cells.

A “molecular beacon” (MB) is an oligonucleotide or PNA which, under appropriate hybridization conditions (e.g., free in solution), self-hybridizes to form a stem and loop structure. The MB has a label and a quencher at the termini of the oligonucleotide or PNA; thus, under conditions that permit intra-molecular hybridization, the label is typically quenched (or otherwise altered) by the quencher. Under conditions where the MB does not display intra-molecular hybridization (e.g., when bound to a target nucleic acid), the MB label is unquenched. A “label” is a moiety that facilitates detection of a molecule. Common labels in the context of the present invention include fluorescent and colorimetric labels. A “quencher” is a moiety that alters a property of the label when it is in proximity to the label. The quencher can actually quench an emission, but it does not have to, i.e., it can simply alter some detectable property of the label, or, when proximal to the label, cause a different detectable property than when not proximal to the label.

The term “nucleic acid” encompasses any physical string of monomer units that can be corresponded to a string of nucleotides, including a polymer of nucleotides (e.g., a typical DNA or RNA polymer), PNAs, modified oligonucleotides (e.g., oligonucleotides comprising bases that are not typical to biological RNA or DNA in solution, such as 2′-O-methylated oligonucleotides), and the like. A nucleic acid can be e.g., single-stranded or double-stranded.

An “oligonucleotide” is a polymer comprising two or more nucleotides. The polymer can additionally comprise non-nucleotide elements such as labels, quenchers, blocking groups, or the like. The nucleotides of the oligonucleotide can be natural or non-natural and can be unsubstituted, unmodified, substituted or modified. The nucleotides can be linked by phosphodiester bonds, or by phosphorothioate linkages, methylphosphonate linkages, boranophosphate linkages, or the like.

A “peptide nucleic acid” (PNA) is a polymer comprising two or more peptide nucleic acid monomers. The polymer can additionally comprise elements such as labels, quenchers, blocking groups, or the like. The monomers of the PNA can be unsubstituted, unmodified, substituted or modified.

A “primer” is a nucleic acid that contains a sequence complementary to a region of a template nucleic acid strand and that primes the synthesis of a strand complementary to the template (or a portion thereof). Primers are typically, but need not be, relatively short, chemically synthesized oligonucleotides. In an amplification, e.g., a PCR amplification, a pair of primers typically define the 5′ ends of the two complementary strands of the target sequence that is amplified.

“Single nucleotide discrimination” refers to discrimination of a target nucleic acid from a variant nucleic acid that differs from the target nucleic acid by as little as a single nucleotide (e.g., substitution or deletion of a single nucleotide, or substitution or deletion of at least two nucleotides).

A “target” or “nucleic acid target” is a region of a nucleic acid that is to be amplified, detected or both.

A “thermostable polymerase” is a polymerase that can tolerate elevated temperatures, at least temporarily, without becoming inactive. For example, a typical thermostable DNA polymerase can tolerate temperatures greater than 90° C. (e.g., 95° C.) for a total time of at least about ten minutes without losing more than about half its activity.

The “Tm” (melting temperature) of a nucleic acid duplex under specified conditions is the temperature at which half of the base pairs are disassociated and half are associated.

“5′ to 3′ nuclease activity” is an enzymatic activity that includes either a 5′ to 3′ exonuclease activity, whereby nucleotides are removed from the 5′ end of a nucleic acid strand (e.g., an oligonucleotide) in a sequential manner; or a 5′ to 3′ endonuclease activity, wherein cleavage occurs more than one nucleotide from the 5′ end; or both. An example of 5′ to 3′ endonuclease activity is the flap endonuclease activity exhibited by the Thermus aquaticus DNA polymerase Taq.

The 5′ to 3′ nuclease activity of a polymerase “substantially lacking 5′ to 3′ nuclease activity” or which is “nuclease-free” is about 20% or less (e.g., 10% or less or 5% or less) than that of the Taq DNA polymerase from Thermus aquaticus under typical reaction conditions (e.g., typical primer extension conditions for the polymerase). Optionally, the nuclease activity of the nuclease-free enzyme can be completely absent, i.e., undetectable under such typical reaction conditions. Thermus aquaticus Taq is described, e.g., in U.S. Pat. No. 4,889,818 and U.S. Pat. No. 5,079,352. Example DNA polymerases substantially lacking 5′ to 3′ nuclease activity include, e.g., any DNA polymerase having undetectable 5′ to 3′ nuclease activity under typical primer extension conditions for that polymerase; the Klenow fragment of E. coli DNA polymerase I; a Thermus aquaticus Taq lacking the N-terminal 235 amino acids (e.g., as described in U.S. Pat. No. 5,616,494); and/or a thermostable DNA polymerase having sufficient deletions (e.g., N-terminal deletions), mutations, or modifications so as to eliminate or inactivate the domain responsible for 5′ to 3′ nuclease activity.

A MB that is “resistant to 5′ to 3′ nuclease activity” is cleaved more slowly under typical reaction conditions for a given 5′ to 3′ nuclease than is a corresponding MB comprising only the four conventional deoxyribonucleotides (A, T, G, and/or C) and phosphodiester linkages.

### DETAILED DESCRIPTION

Methods for performing combined amplification (e.g., PCR amplification) and detection of nucleic acid targets are provided, along with attendant compositions, systems, apparatus and kits. The present invention uses nuclease-free DNA polymerase during asymmetric amplification (e.g., asymmetric PCR amplification) of a nucleic acid target. Asymmetric amplification using a nuclease-free polymerase provides dramatic improvements in MB signal intensity and quantitative detection, as described in more detail herein.

Asymmetric PCR strategies have been used in the past to enhance MB signal intensity. For example, Poddar (2000) “Symmetric vs. asymmetric PCR and molecular beacon probe in the detection of a target gene of adenovirus” Molecular and Cellular Probes 14: 25-32 describe a moderate improvement in MB signal intensity following asymmetric PCR as compared to standard symmetric PCR. However, Poddar did not use a nuclease-free DNA polymerase for the asymmetric PCR and, thus, the MB signal improvement observed for the asymmetric PCR of Poddar is far less than that observed in the present invention. The present invention provides for dramatically improved MB signal intensity using an asymmetric PCR amplification strategy, e.g., in conjunction with a nuclease-free polymerase. Other features that are also dramatically improved as compared to the prior art include improved signal to noise ratios and improved MB sensitivity.

One aspect of the invention is the discovery that standard PCR reactions using standard MBs do not operate as supposed. That is, most forms of DNA polymerase in commercial use for PCR (e.g., Taq and many common commercial variants) have a nuclease activity (e.g., a 5′-3′ nuclease activity). This nuclease activity results in degradation of the MB upon binding of the MB to a target, resulting in a release of the MB label from the fluorophore. This cleavage results in signal generation, which is interpreted as MB binding, but at signal formation rates that are not as one would predict from first principles. This renders inaccurate many quantitative aspects of real time amplicon detection with MBs. The degradation of the MBs also substantially limit the ability of previously used asymmetric PCR strategies, such as those described by Poddar, from showing substantial improvement in MB signal or real-time hybridization properties.

It is also worth noting that at least one alternate approach of the invention shows similar results to the use of nuclease-free DNA polymerases in the asymmetric PCR reactions that are monitored using MBs as described herein. That is, asymmetric PCR strategies can also be used with MBs that are themselves nuclease resistant, whether the polymerase which is used for PCR is nuclease-free or not. For example, MBs can be made from modified nucleic acids (e.g., using 2-O-methylated residues or phosphorothioate linkages or other nuclease resistant MBs), or MBs can be treated to increase MB nuclease resistance, e.g., via carbothoxylization, or MBs can simply be made using peptide nucleic acids (PNAs) in place of standard nucleic acids in the MBs. Combinations of typical nuclease resistance modification strategies can also be used, e.g., 2′O methyl phosphoramidite reagents can be used in place of standard reagents and phosphothiolation with sufurization agents can also be employed in generating nuclease resistant beacons.

The methods of this invention can be useful in essentially any application wherein molecular beacons are used to detect the products of an amplification reaction. For example, the methods can be used in monitoring gene expression; genotyping; mutation detection; infectious disease detection; species, allele, and/or single nucleotide polymorphism (SNP) detection; and other diagnostic assays. The increased sensitivity provided by the methods makes them particularly useful for SNP discrimination, allele discrimination, strain identification, and other similar applications wherein a nucleic acid target is discriminated on the basis of a single nucleotide mismatch to the target-recognition sequence of the molecular beacon, and/or applications in which the Tm of the MB target-recognition sequence-target duplex must be close to (e.g., a few degrees above) the temperature at which annealing of the MB and target is monitored.

Amplification with Nuclease-Free Polymerase

One aspect of the present invention provides new asymmetric amplification strategies (e.g., asymmetric PCR strategies) using nuclease-free polymerase to enhance MB-mediated target detection. The methods facilitate combined amplification and detection of a nucleic acid target. In the methods, a molecular beacon, a first primer, a second primer, a template nucleic acid, and a polymerase substantially lacking 5′ to 3′ nuclease activity are provided. The molecular beacon comprises a region of complementarity to a first region of a first strand of a nucleic acid target. The first primer comprises a region of identity with a second region of the first strand of the nucleic acid target, and the second primer comprises a region of complementarity to a third region of the first strand of the nucleic acid target. The third region is 3′ of the first region, and the first region is 3′ of the second region (that is, the two primers typically define the 5′ ends of the two complementary strands of a double-stranded product of the amplification). The first primer is provided at a concentration that is at least about 1.3 times that of the second primer. The template nucleic acid comprises the first strand of the nucleic acid target, a second strand of the nucleic acid target that is complementary to the first strand, or both. The target nucleic acid is amplified by subjecting the template nucleic acid, the first and second primers, the molecular beacon, and the polymerase to cycles comprising denaturation, annealing, and extension steps. A signal from the molecular beacon is detected at at least one time point during or after the cycles.

The first primer is provided a concentration that is at least about 1.3 times (e.g., at least about two times) that of the second primer. In one class of embodiments, the first primer is provided at a concentration that is at least about three times (e.g., at least about 3.5 times, at least about four times, at least about five times, or more) the concentration of the second primer. Use of an excess of one primer results in asymmetric amplification and production of more of the strand into which the first primer is incorporated and to which the MB (i.e., the target-recognition sequence of the MB) is complementary, enhancing MB-mediated target detection.

Amplification of nucleic acid targets by cyclical polymerase-mediated extension of primers (e.g., PCR amplification) is well known in the art. During the denaturation step, the template (if double-stranded) and/or the double-stranded extension product of a previous cycle is denatured (e.g., by a chemical denaturant or by thermal denaturation). One or both primers anneal to a complementary strand of the template during the annealing step. Annealing can be accomplished, for example, by decreasing the concentration of chemical denaturant or decreasing the temperature. During the extension step, the polymerase catalyzes template-dependent extension of the primers, in the presence of deoxyribonucleoside triphosphates, an aqueous buffer, appropriate salts and metal cations, and the like, to form a double-stranded extension product comprising the nucleic acid target.

In one class of embodiments, the cycles of denaturation, annealing, and extension steps comprise thermal cycles. For example, the thermal cycles can comprise cycles of denaturation at temperatures greater than about 90° C., annealing at 50-75° C., and extension at 72-78° C. A thermostable polymerase is thus preferred. For example, the thermostable polymerase can be a DNA polymerase or modified form thereof from a Thermus species (e.g., Thermus aquaticus, Thermus ruber, Thermus flavus, Thermus thermophilus, or Thermus lacteus). Thermostable polymerases lacking 5′ to 3′ nuclease activity are commercially available, e.g., Titanium® Taq (Clontech, www.clontech.com), KlenTaq DNA polymerase (Sigma-Aldrich, www.sigma-aldrich.com), Vent® and DeepVent® DNA polymerase (New England Biolabs, www.neb.com), and Tgo DNA polymerase (Roche, www.roche-applied-science.com).

The polymerase is substantially lacking 5′ to 3′ nuclease activity. That is, the polymerase has a 5′ to 3′ nuclease activity that is about twenty percent or less than that of the Thermus aquaticus Taq DNA polymerase under typical reaction conditions (e.g., typical primer extension conditions for the polymerase, e.g., typical PCR conditions). In other words, the 5′ to 3′ nuclease activity of the polymerase is about one-fifth, or less than about one-fifth, the 5′ to 3′ nuclease activity of Taq. In other embodiments, the polymerase has a 5′ to 3′ nuclease activity that is ten percent or less (e.g., five percent or less) than that of Taq under typical reaction conditions. Optionally, the polymerase has no detectable 5′ to 3′ nuclease activity under typical reaction conditions (e.g., typical PCR conditions).

In one class of embodiments, the signal from the MB is detected during the annealing step of each cycle (e.g., at at least one time point during the annealing step, e.g., where the time point is defined by the achievement of a preselected temperature). In this class of embodiments, the MB (i.e., the target-recognition loop of the MB) can bind to the first strand of the nucleic acid target during the annealing step. As described briefly herein, binding of the molecular beacon to the target results in a detectable signal from the MB (e.g., a characteristic fluorescent emission, or a change in absorption spectrum). The MB can melt off the target prior to or during the extension step, and thus not interfere with extension of the second primer.

In one class of embodiments, a fluorescent emission from the molecular beacon is detected at at least one time point during or after the cycles (e.g., during the annealing step of each cycle). In certain embodiments, the intensity of the fluorescent emission is measured.

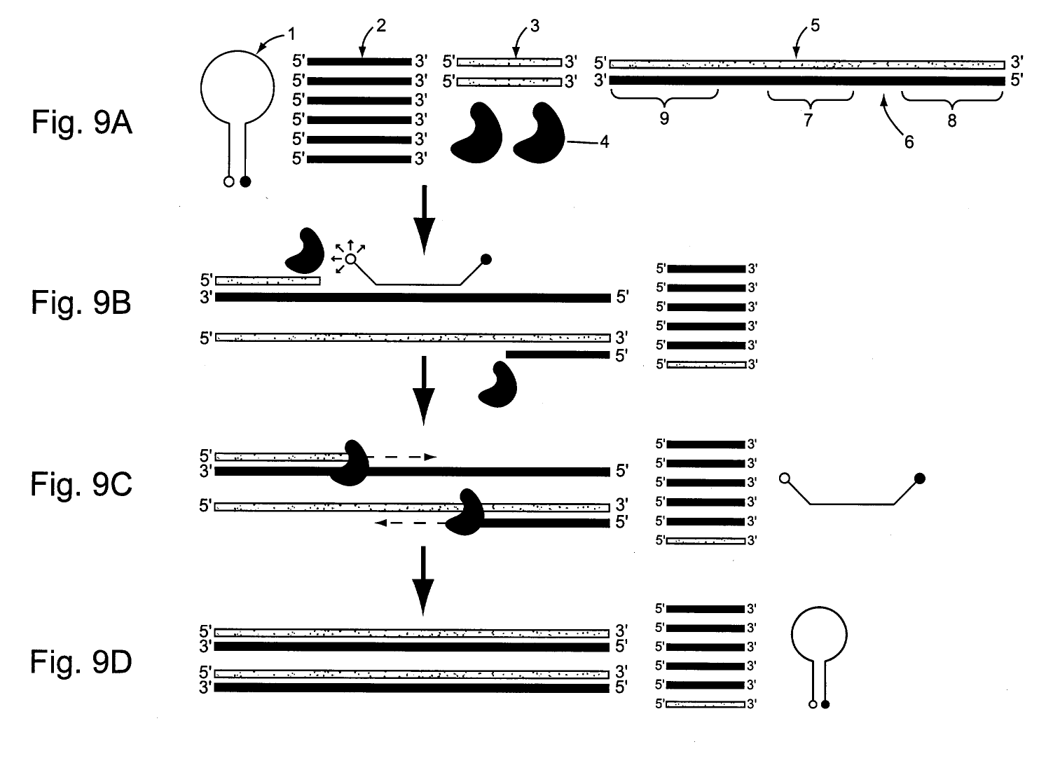

An example of an asymmetric PCR amplification using nuclease-free polymerase in which the MB binds to the nucleic acid target during the annealing step is schematically illustrated in FIG. 9. Panel A depicts MB 1 in its hairpin conformation, in which the fluorophore (open circle) is quenched by the quencher (filled circle); first primer 2, which is present in excess (e.g., at least threefold excess as depicted) of second primer 3; polymerase 4 substantially lacking 5′ to 3′ nuclease activity; and double-stranded template 5 and 6 comprising the target. As depicted, the template is identical to the double-stranded extension product of each cycle, but as will be evident one of skill in the art, the template initially provided can be, e.g., single-stranded (comprising either strand) or double-stranded and can contain additional sequences 5′ and/or 3′ of the nucleic acid target that are not amplified. As illustrated, the loop (the target-recognition sequence) of the MB is complementary to first region 7 of first strand 6 of the target, the first primer is identical to second region 8 of first strand 6, and the second primer is complementary to third region 9 of first strand 6. The double-stranded template (or a double-stranded extension product from a previous cycle) is denatured, e.g., at temperatures greater than about 90° C. The temperature is decreased (e.g., to 50-75° C.), and one or both primers and the MB anneal to their respective strands of the target. As depicted in Panel B, when the target recognition sequence of the molecular beacon is bound to its complementary sequence in the target, the fluorophore and quencher are separated, resulting in a measurable signal (e.g., an increase in fluorescence) from the MB. As illustrated in Panel C, the MB typically disassociates from the target at the higher temperatures (e.g., 72-78° C.) used for extension of one or both primers by the polymerase. Panel D depicts the double-stranded extension products, which can be used as template in another cycle. FIG. 9 depicts annealing and extension of both the first and second primers; however, as the second primer is depleted and its concentration becomes limiting, in many instances only the first primer will be available for annealing and extension, resulting in the production of more of first strand 6 than second strand 5.

The nucleic acid target can be essentially any nucleic acid. For example, the nucleic acid target to be amplified and/or detected can be single-stranded or double-stranded and can comprise a DNA, a genomic DNA, a cDNA, a synthetic oligonucleotide, an RNA, an mRNA, or a viral RNA genome, to list only a few. The nucleic acid can be derived from any source, including but not limited to: a human; an animal; a plant; a bacterium; a virus; cultured cells or culture medium; a tissue or fluid, e.g., from a patient, such as skin, blood, sputum, urine, stool, semen, or spinal fluid; a tumor; a biopsy; and/or the like.

The template nucleic acid comprising the target nucleic acid can be e.g. any single-stranded or double-stranded DNA. For example, in one class of embodiments, the template nucleic acid is a single-stranded DNA product of a reverse transcription reaction. Molecular beacons can thus be conveniently used to detect RNA targets by rt-PCR (including quantitative rt-PCR).

Molecular beacons can also be used, e.g., for in situ PCR. Thus, in one class of embodiments, the template nucleic acid is located within one or more fixed cells. The signal from the MB can optionally be detected in a manner that locates the MB within the individual cells or individual subcellular structures that initially contained the template nucleic acid.

The methods can be performed, e.g., in solution. In other embodiments, one or more of the molecular beacon, primers, template, or polymerase are not free in solution. For example, in certain embodiments, the template nucleic acid is bound (e.g., electrostatically bound or covalently bound, e.g., directly or via a linker) to a matrix. Example matrices include, but are not limited to, a surface, a beaded support, a cast or solution insoluble polymer, or a gel. See, e.g., U.S. Pat. No. 6,441,152 (Aug. 27, 2002) to Johansen et al. entitled “Methods, kits and compositions for the identification of nucleic acids electrostatically bound to matrices.”

In another class of embodiments, the methods facilitate the amplification and detection of two or more nucleic acid targets simultaneously (e.g., by multiplex PCR). In this class of embodiments, two or more molecular beacons, each of which comprises a region of complementarity to a strand of a different nucleic acid target, are provided. A pair of primers (a first and second primer) are provided for each different nucleic acid target, wherein each first primer is provided at a concentration that is at least about 1.3 times (e.g., at least about two times, at least about three times, or more) that of the corresponding second primer. A template nucleic acid for each different nucleic acid target is provided, and each target nucleic acid is amplified. A signal from each of the two or more molecular beacons is detected. The signals from the different MBs are typically distinguishable from each other, such that information about each different target can be acquired. For example, each MB can fluoresce at a different wavelength, or the MBs can be spatially resolved. In certain embodiments, the template nucleic acids form an array on a matrix. In the array, each template nucleic acid is bound (e.g., electrostatically or covalently bound) to the matrix at a unique location. Methods of making, using, and analyzing such arrays (e.g., microarrays) are well known in the art.

As mentioned previously, the methods are particularly useful for applications (e.g., SNP detection) in which the target nucleic acid is to be discriminated from one or more similar variants (e.g., a nucleic acid with a single nucleotide substitution). Thus, in one class of embodiments, the method is used for single nucleotide discrimination. For such applications, the Tm of the MB target-recognition sequence-target duplex is greater than, and preferably close to (e.g., a few degrees higher than), the temperature at which association of the MB and target is monitored. Since mismatched MB target-recognition sequence-variant sequence hybrids have a lower Tm than do perfectly complementary MB target-recognition sequence-target hybrids, a detection temperature can be chosen that is less than the Tm of the perfectly complementary MB target-recognition sequence-target duplex and greater than the Tm of the mismatched (e.g., singly mismatched) MB target recognition sequence-variant duplex. That is, the signal (e.g., fluorescence) from the molecular beacon can be monitored under conditions in which less than perfect complementarity between the target recognition sequence of the MB and a nucleic acid strand results in failure of the MB to hybridize to that strand. Details regarding design of MBs for single nucleotide discrimination are established in the literature, e.g., Marras et al. (1999) “Multiplex detection of single-nucleotide variation using molecular beacons” Genet. Anal. Biomol. Eng. 14:151-156, and Mhlanga et al. (2001) “Using molecular beacons to detect single-nucleotide polymorphisms with real-time PCR” Methods 25:463-471.

Amplification and Detection with Nuclease-Resistant Molecular Beacons

Another aspect of the present invention provides new asymmetric amplification strategies (e.g., asymmetric PCR strategies) using nuclease-resistant MBs to enhance MB-mediated target detection. The methods facilitate combined amplification and detection of a nucleic acid target. In the methods, a molecular beacon, a first primer, a second primer, a template nucleic acid, and a polymerase are provided. The molecular beacon comprises a region of complementarity to a first region of a first strand of a nucleic acid target, and the MB is resistant to 5′ to 3′ nuclease activity. The first primer comprises a region of identity with a second region of the first strand of the nucleic acid target, and the second primer comprises a region of complementarity to a third region of the first strand of the nucleic acid target. The third region is 3′ of the first region, and the first region is 3′ of the second region (that is, the two primers typically define the 5′ ends of the two complementary strands of a double-stranded product of the amplification). The first primer is provided at a concentration that is at least about 1.3 times that of the second primer. The template nucleic acid comprises the first strand of the nucleic acid target, a second strand of the nucleic acid target that is complementary to the first strand, or both. The target nucleic acid is amplified by subjecting the template nucleic acid, the first and second primers, the molecular beacon, and the polymerase to cycles comprising denaturation, annealing, and extension steps. A signal from the molecular beacon is detected at at least one time point during or after the cycles.

The first primer is provided a concentration that is at least about 1.3 times (e.g., at least about two times) that of the second primer. In one class of embodiments, the first primer is provided at a concentration that is at least about three times (e.g., at least about 3.5 times, at least about four times, at least about five times, or more) the concentration of the second primer. Use of an excess of one primer results in asymmetric amplification and production of more of the strand into which the first primer is incorporated and to which the MB (i.e., the target-recognition sequence of the MB) is complementary, enhancing MB-mediated target detection.

Amplification of nucleic acid targets by cyclical polymerase-mediated extension of primers (e.g., PCR amplification) is well known in the art. During the denaturation step, the template (if double-stranded) and/or the double-stranded extension product of a previous cycle is denatured (e.g., by a chemical denaturant or by thermal denaturation). One or both primers anneal to a complementary strand of the template during the annealing step. Annealing can be accomplished, for example, by decreasing the concentration of chemical denaturant or decreasing the temperature. During the extension step, the polymerase catalyzes template-dependent extension of the primers, in the presence of deoxyribonucleoside triphosphates, an aqueous buffer, appropriate salts and metal cations, and the like, to form a double-stranded extension product comprising the nucleic acid target.

In one class of embodiments, the cycles of denaturation, annealing, and extension steps comprise thermal cycles. For example, the thermal cycles can comprise cycles of denaturation at temperatures greater than about 90° C., annealing at 50-75° C., and extension at 72-78° C. A thermostable polymerase is thus preferred. A variety of thermostable DNA polymerases (e.g., Taq) are commercially available. The polymerase can have or can substantially lack (e.g., have undetectable) 5′ to 3′ nuclease activity.

In one class of embodiments, the signal from the MB is detected during the annealing step of each cycle (e.g., at at least one time point during the annealing step, e.g., where the time point is defined by the achievement of a preselected temperature). In this class of embodiments, the MB (i.e., the target-recognition loop of the MB) can bind to the first strand of the nucleic acid target during the annealing step. As described briefly herein, binding of the molecular beacon to the target results in a detectable signal from the MB (e.g., a characteristic fluorescent emission, or a change in absorption spectrum). The MB can melt off the target prior to or during the extension step, and thus not interfere with extension of the second primer.

In one class of embodiments, a fluorescent emission from the molecular beacon is detected at at least one time point during or after the cycles (e.g., during the annealing step of each cycle). In certain embodiments, the intensity of the fluorescent emission is measured.

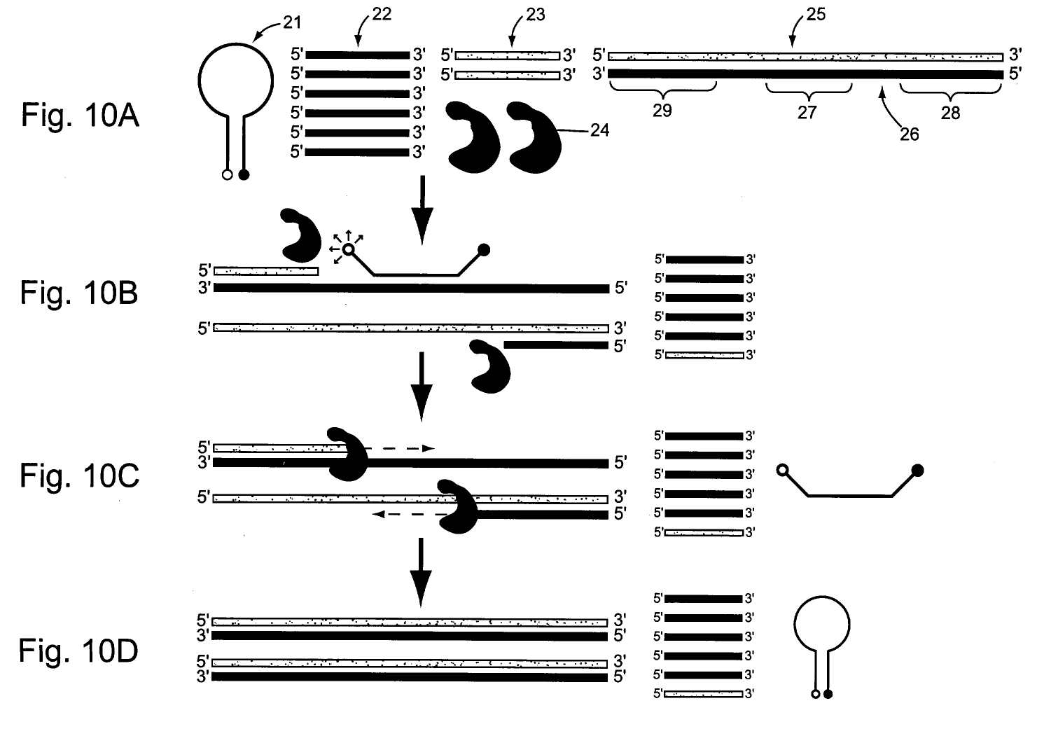

An example of an asymmetric PCR amplification in which the nuclease-resistant MB binds to the nucleic acid target during the annealing step is schematically illustrated in FIG. 10. Panel A depicts nuclease-resistant MB 21 in its hairpin conformation, in which the fluorophore (open circle) is quenched by the quencher (filled circle); first primer 22, which is present in excess (e.g., at least threefold excess as depicted) of second primer 23; polymerase 24 (optionally, a polymerase substantially lacking 5′ to 3′ nuclease activity); and double-stranded template 25 and 26 comprising the target. As depicted, the template is identical to the double-stranded extension product of each cycle, but as will be evident one of skill in the art, the template initially provided can be, e.g., single-stranded (comprising either strand) or double-stranded and can contain additional sequences 5′ and/or 3′ of the nucleic acid target that are not amplified. As illustrated, the loop (the target-recognition sequence) of the MB is complementary to first region 27 of first strand 26 of the target, the first primer is identical to second region 28 of first strand 26, and the second primer is complementary to third region 29 of first strand 26. The double-stranded template (or a double-stranded extension product from a previous cycle) is denatured, e.g., at temperatures greater than about 90° C. The temperature is decreased (e.g., to 50-75° C.), and one or both primers and the MB anneal to their respective strands of the target. As depicted in Panel B, when the target recognition sequence of the molecular beacon is bound to its complementary sequence in the target, the fluorophore and quencher are separated, resulting in a measurable signal (e.g., an increase in fluorescence) from the MB. As illustrated in Panel C, the MB typically disassociates from the target at the higher temperatures (e.g., 72-78° C.) used for extension of one or both primers by the polymerase. Panel D depicts the double-stranded extension products, which can be used as template in another cycle. FIG. 10 depicts annealing and extension of both the first and second primers; however, as the second primer is depleted and its concentration becomes limiting, in many instances only the first primer will be available for annealing and extension, resulting in the production of more of first strand 26 than second strand 25.

The nucleic acid target can be essentially any nucleic acid. For example, the nucleic acid target to be amplified and/or detected can be single-stranded or double-stranded and can comprise a DNA, a genomic DNA, a cDNA, a synthetic oligonucleotide, an RNA, an mRNA, or a viral RNA genome, to list only a few. The nucleic acid can be derived from any source, including but not limited to: a human; an animal; a plant; a bacterium; a virus; cultured cells or culture medium; a tissue or fluid, e.g., from a patient, such as skin, blood, sputum, urine, stool, semen, or spinal fluid; a tumor; a biopsy; and/or the like.

The template nucleic acid comprising the target nucleic acid can be e.g. any single-stranded or double-stranded DNA. For example, in one class of embodiments, the template nucleic acid is a single-stranded DNA product of a reverse transcription reaction. Molecular beacons can thus be conveniently used to detect RNA targets by rt-PCR (including quantitative rt-PCR).

Molecular beacons can also be used, e.g., for in situ PCR. Thus, in one class of embodiments, the template nucleic acid is located within one or more fixed cells. The signal from the MB can optionally be detected in a manner that locates the MB within the individual cells or individual subcellular structures that initially contained the template nucleic acid.

The methods can be performed, e.g., in solution. In other embodiments, one or more of the molecular beacon, primers, template, or polymerase are not free in solution. For example, in certain embodiments, the template nucleic acid is bound (e.g., electrostatically bound or covalently bound, e.g., directly or via a linker) to a matrix. Example matrices include, but are not limited to, a surface, a beaded support, a cast or solution insoluble polymer, or a gel. See, e.g., U.S. Pat. No. 6,441,152 (Aug. 27, 2002) to Johansen et al. entitled “Methods, kits and compositions for the identification of nucleic acids electrostatically bound to matrices.”

In another class of embodiments, the methods facilitate the amplification and detection of two or more nucleic acid targets simultaneously (e.g., by multiplex PCR). In this class of embodiments, two or more molecular beacons, each of which comprises a region of complementarity to a strand of a different nucleic acid target and each of which is resistant to 5′ to 3′ nuclease activity, are provided. A pair of primers (a first and second primer) are provided for each different nucleic acid target, wherein each first primer is provided at a concentration that is at least about 1.3 times (e.g., at least about two times, at least about three times, or more) that of the corresponding second primer. A template nucleic acid for each different nucleic acid target is provided, and each target nucleic acid is amplified. A signal from each of the two or more molecular beacons is detected. The signals from the different MBs are typically distinguishable from each other, such that information about each different target can be acquired. For example, each MB can fluoresce at a different wavelength, or the MBs can be spatially resolved. In certain embodiments, the template nucleic acids form an array on a matrix. In the array, each template nucleic acid is bound (e.g., electrostatically or covalently bound) to the matrix at a unique location. Methods of making, using, and analyzing such arrays (e.g., microarrays) are well known in the art.

As mentioned previously, the methods are particularly useful for applications (e.g., SNP detection) in which the target nucleic acid is to be discriminated from one or more similar variants (e.g., a nucleic acid with a single nucleotide substitution). Thus, in one class of embodiments, the method is used for single nucleotide discrimination. For such applications, the Tm of the MB target-recognition sequence-target duplex is greater than, and preferably close to (e.g., a few degrees higher than), the temperature at which association of the MB and target is monitored. Since mismatched MB target-recognition sequence-variant sequence hybrids have a lower Tm than do perfectly complementary MB target-recognition sequence-target hybrids, a detection temperature can be chosen that is less than the Tm of the perfectly complementary MB target-recognition sequence-target duplex and greater than the Tm of the mismatched (e.g., singly mismatched) MB target recognition sequence-variant duplex. That is, the signal (e.g., fluorescence) from the molecular beacon can be monitored under conditions in which less than perfect complementarity between the target recognition sequence of the MB and a nucleic acid strand results in failure of the MB to hybridize to that strand. Details regarding design of MBs for single nucleotide discrimination are established in the literature, e.g., Marras et al. (1999) “Multiplex detection of single-nucleotide variation using molecular beacons” Genet. Anal. Biomol. Eng. 14:151-156, and Mhlanga et al. (2001) “Using molecular beacons to detect single-nucleotide polymorphisms with real-time PCR” Methods 25:463-471.

A variety of nuclease-resistant MBs can be created, e.g., comprising modified nucleotides or modified internucleotide linkages such as those used in the synthesis of antisense oligonucleotides. In one class of embodiments, the molecular beacon comprises a peptide nucleic acid (PNA). In another class of embodiments, the MB comprises one or more 2′-O-methyl nucleotides. For example, a MB comprising standard deoxyribonucleotides can also comprise one or more 2′-O-methyl nucleotides (e.g., at its 5′ end), or a MB can consist entirely of 2′-O-methyl nucleotides. In some embodiments, the molecular beacon comprises one or more phosphorothioate linkages (oligonucleotides comprising such linkages are sometimes called S-oligos). A MB can comprise, e.g., only phosphorothioate linkages or a mixture of phosphodiester and phosphorothioate linkages. In other embodiments, the MB comprises one or more methylphosphonate linkages, one or more boranophosphate linkages, or the like. Combinations of typical nuclease resistance modification strategies can also be employed; for example, a nuclease resistant MB can comprise both 2′-O-methyl nucleotides and phosphorothioate linkages.

Compositions, Systems, Devices and Kits

The present invention also includes compositions, systems, devices and kits, e.g., for practicing the methods herein or which are produced by the methods herein.

For Nuclease-Free Amplification

In one general class of embodiments, the invention provides a composition comprising a molecular beacon, a first primer, a second primer, and a polymerase substantially lacking 5′ to 3′ nuclease activity. The molecular beacon comprises a region of complementarity to a first region of a first strand of a nucleic acid target. The first primer comprises a region of identity with a second region of the first strand of the nucleic acid target, and the second primer comprising a region of complementarity to a third region of the first strand of the nucleic acid target. The third region is 3′ of the first region, and the first region is 3′ of the second region. The first primer is present at a concentration that is at least about 1.3 times (e.g., at least about two times) that of the second primer.

In one class of embodiments, the first primer is present at a concentration that is at least about three times (e.g., at least about 3.5 times, at least about four times, at least about five times, or more) the concentration of the second primer.

The composition can further comprise a template nucleic acid, wherein the template comprises the first strand of the nucleic acid target, a second strand of the nucleic acid target that is complementary to the first strand, or both.

The polymerase can be a thermostable polymerase, e.g., a DNA polymerase, or a modified form thereof, from a Thermus species (commercially available examples include, e.g., Titanium® Taq (Clontech, www.clontech.com), KlenTaq DNA polymerase (Sigma-Aldrich, www.sigma-aldrich.com), Vent® and DeepVent® DNA polymerase (New England Biolabs, www.neb.com), and Tgo DNA polymerase (Roche, www.roche-applied-science.com)).

The polymerase is substantially lacking 5′ to 3′ nuclease activity. That is, the polymerase has a 5′ to 3′ nuclease activity that is about twenty percent or less than that of the Thermus aquaticus Taq DNA polymerase under typical reaction conditions (e.g., typical primer extension conditions for the polymerase, e.g., typical PCR conditions). In other words, the 5′ to 3′ nuclease activity of the polymerase is about one-fifth, or less than about one-fifth, the 5′ to 3′ nuclease activity of Taq. In other embodiments, the polymerase has a 5′ to 3′ nuclease activity that is ten percent or less (e.g., five percent or less) than that of Taq under typical reaction conditions. Optionally, the polymerase has no detectable 5′ to 3′ nuclease activity under typical reaction conditions (e.g., typical PCR conditions).

The composition can be formed, e.g., in solution, or at one or more positions on an array.

In one aspect, the invention includes systems and devices for use of the compositions, e.g., according to the methods herein. In one class of embodiments, the composition is contained in a thermal cycler (e.g., in one or more sample tubes or one or more wells of a multiwell plate, in a reaction region of a thermal cycler, e.g., an automated thermal cycler). The system can include, e.g., a computer with appropriate software for controlling the operation of the thermal cycler (e.g., temperature and duration of each step, ramping between steps, and/or number of cycles) coupled to the thermal cycler. Similarly, the system can include a detector coupled to the thermal cycler and/or computer (e.g., for measuring the fluorescence spectrum and/or intensity from one or more wells of a multiwell plate contained in the reaction region of the thermal cycler after excitation by laser light source).

The computer typically includes appropriate software for receiving user instructions, either in the form of user input into a set of parameter fields, e.g., in a GUI, or in the form of preprogrammed instructions, e.g., preprogrammed for a variety of different specific operations. The software optionally converts these instructions to appropriate language for instructing the operation of the thermal cycler to carry out the desired operation. The computer can also receive data from the thermal cycler and/or detector regarding fluorescent intensity, cycle completion or the like and can interpret the data, provide it to a user in a human readable format, or use that data to initiate further operations (e.g., additional thermal cycles), in accordance with any programming by the user.

One class of embodiments provides a kit, comprising the molecular beacon, the first and second primers, and the polymerase, packaged in one or more containers. The kit can further comprise one or more of: a buffer, a standard target for calibrating a detection reaction, instructions for using the components to detect and/or quantitate the nucleic acid target, or packaging materials.

For Amplification and Detection with Nuclease-Resistant Molecular Beacons

In another general class of embodiments, the invention provides a composition comprising a molecular beacon, a first primer, and a second primer. The molecular beacon comprises a region of complementarity to a first region of a first strand of a nucleic acid target, and the MB is resistant to 5′ to 3′ nuclease activity. The first primer comprises a region of identity with a second region of the first strand of the nucleic acid target, and the second primer comprising a region of complementarity to a third region of the first strand of the nucleic acid target. The third region is 3′ of the first region, and the first region is 3′ of the second region. The first primer is present at a concentration that is at least about 1.3 times (e.g., at least about two times) that of the second primer.

The nuclease resistant molecular beacon can comprise, for example, a peptide nucleic acid, one or more 2′-O-methyl nucleotides, and/or one or more phosphorothioate linkages.

In one class of embodiments, the first primer is present at a concentration that is at least about three times (e.g., at least about 3.5 times, at least about four times, at least about five times, or more) the concentration of the second primer.

The composition can further comprise a template nucleic acid, wherein the template comprises the first strand of the nucleic acid target, a second strand of the nucleic acid target that is complementary to the first strand, or both. Alternatively or in addition, the composition can further comprise a polymerase. In some embodiments, the polymerase is a thermostable polymerase, e.g., a DNA polymerase, or a modified form thereof, from a Thermus species, e.g., Taq or Titanium® Taq.

The composition can be formed, e.g., in solution, or at one or more positions on an array.

In one aspect, the invention includes systems and devices for use of the compositions, e.g., according to the methods herein. In one class of embodiments, the composition is contained in a thermal cycler (e.g., in one or more sample tubes or one or more wells of a multiwell plate, in a reaction region of a thermal cycler, e.g., an automated thermal cycler). The system can include, e.g., a computer with appropriate software for controlling the operation of the thermal cycler (e.g., temperature and duration of each step, ramping between steps, and/or number of cycles) coupled to the thermal cycler. Similarly, the system can include a detector coupled to the thermal cycler and/or computer (e.g., for measuring the fluorescence spectrum and/or intensity from one or more wells of a multiwell plate contained in the reaction region of the thermal cycler after excitation by laser light source).

The computer typically includes appropriate software for receiving user instructions, either in the form of user input into a set of parameter fields, e.g., in a GUI, or in the form of preprogrammed instructions, e.g., preprogrammed for a variety of different specific operations. The software optionally converts these instructions to appropriate language for instructing the operation of the thermal cycler to carry out the desired operation. The computer can also receive data from the thermal cycler and/or detector regarding fluorescent intensity, cycle completion or the like and can interpret the data, provide it to a user in a human readable format, or use that data to initiate further operations (e.g., additional thermal cycles), in accordance with any programming by the user.

One class of embodiments provides a kit, comprising the molecular beacon, and the first and second primers, packaged in one or more containers. The kit can further comprise one or more of: a polymerase, a buffer, a standard target for calibrating a detection reaction, instructions for using the components to detect and/or quantitate the nucleic acid target, or packaging materials.

Synthesis and Use of Molecular Beacons

In brief, in a molecular beacon, a central target-recognition sequence is flanked by arms that hybridize to one another when the probe is not hybridized to a target strand, forming a “hairpin” structure, in which the target-recognition sequence (which is sometimes referred to as the “probe sequence”) is in the single-stranded loop of the hairpin structure, and the arm sequences form a double-stranded stem hybrid. Molecular beacon probes can typically have target recognition sequences of, e.g., about 7-140 nucleotides in length and arms that form a stem hybrid, or “stem duplex” of e.g., about 3-25 nucleotides in length. Modified nucleotides and modified nucleotide linkages may be used for MB construction, even including, e.g., peptide nucleic acid (PNAs).

Operation of the MB is rather straightforward. When the probe sequence hybridizes to a target, a relatively rigid helix is formed, causing the stem hybrid to unwind and forcing the arms of the MB apart. A label/quencher pair, such as the fluorophore EDANS and the quencher DABCYL, are attached to the arms, e.g., by alkyl spacers. When the MB is not hybridized to a target strand, the fluorophore's emission is quenched due to proximity of the fluorophore and quencher. When the MB is hybridized to a target strand, the fluorophore and quencher are separated and the fluorophore's emission is not quenched. Thus, emitted fluorescence signals the presence, in real time, of target strands being hybridized to the MB.

MBs can incorporate any of a variety of fluorophore/quencher combinations, using e.g., fluorescence resonance energy transfer (FRET)-based quenching, non-FRET based quenching, or wavelength-shifting harvester molecules. Example combinations include terbium chelate and TRITC (tetrarhodamine isothiocyanate), europium cryptate and Allophycocyanin, fluorescein and tetramethylrhodamine, IAEDANS and fluorescein, EDANS and DABCYL, fluorescein and DABCYL, fluorescein and fluorescein, BODIPY FL and BODIPY FL, and fluorescein and QSY 7 dye. Nonfluorescent acceptors such as DABCYL and QSY 7 and QSY 33 dyes have the particular advantage of eliminating background fluorescence resulting from direct (i.e., nonsensitized) acceptor excitation. A variety of probes incorporating fluorescent donor-nonfluorescent acceptor combinations have been developed for detection of nucleic acid hybridization events. See e.g., Haugland (1996) Handbook of Fluorescent Probes and Research Chemicals published by Molecular Probes, Inc., Eugene, Oreg. e.g., at chapter 13) or a more current on-line (www.probes.com) or CD-ROM version of the Handbook (available from Molecular Probes, Inc.). Detectable signals from such molecular beacons include changes in fluorescence and/or changes in absorption spectra.

Absorption by or fluorescent emissions from MBs can be detected by essentially any method known in the art. In the context of real time PCR, for example, fluorescent emissions can be conveniently detected during the amplification by use of a commercially available integrated system such as, e.g., the ABI Prism® 7700 sequence detection system from Applied Biosystems (www.appliedbiosystems.com), or the iCycler iQ® real-time PCR detection system from Bio-Rad (www.biorad.com).

MBs can be synthesized using conventional methods. For example, oligos or PNAs can be synthesized on commercially available automated oligonucleotide/PNA synthesis machines using standard methods. Labels can be attached to the oligos or PNAs either during automated synthesis or by post-synthetic reactions which have been described before see, e.g., Tyagi and Kramer (1996) “Molecular beacons: probes that fluoresce upon hybridization” Nature Biotechnology 14:303-308 and U.S. Pat. No. 6,037,130 to Tyagi et al (Mar. 14, 2000), entitled “Wavelength-shifting probes and primers and their use in assays and kits.” and U.S. Pat. No. 5,925,517 (Jul. 20, 1999) to Tyagi et al. entitled “Detectably labeled dual conformation oligonucleotide probes, assays and kits.” Additional details on synthesis of functionalized oligos can be found in Nelson, et al. (1989) “Bifunctional Oligonucleotide Probes Synthesized Using A Novel CPG Support Are Able To Detect Single Base Pair Mutations” Nucleic Acids Research 17:7187-7194.

Labels/quenchers can be introduced to the oligonucleotides or PNAs, e.g., by using a controlled-pore glass column to introduce, e.g., the quencher (e.g., a 4-dimethylaminoazobenzene-4′-sulfonyl moiety (DABSYL). For example, the quencher can be added at the 3′ end of oligonucleotides during automated synthesis; a succinimidyl ester of 4-(4′-dimethylaminophenylazo)benzoic acid (DABCYL) can be used when the site of attachment is a primary amino group; and 4-dimethylaminophenylazophenyl-4′-maleimide (DABMI) can be used when the site of attachment is a sulphydryl group. Similarly, fluorescein can be introduced in the oligos, either using a fluorescein phosphoramidite that replaces a nucleoside with fluorescein, or by using a fluorescein dT phosphoramadite that introduces a fluorescein moiety at a thymidine ring via a spacer. To link a fluorescein moiety to a terminal location, iodoacetoamidofluorescein can be coupled to a sulphydryl group. Tetrachlorofluorescein (TET) can be introduced during automated synthesis using a 5′-tetrachloro-fluorescein phosphoramadite. Other reactive fluorophore derivatives and their respective sites of attachment include the succinimidyl ester of 5-carboxyrhodamine-6G (RHD) coupled to an amino group; an iodoacetamide of tetramethylrhodamine coupled to a sulphydryl group; an isothiocyanate of tetramethylrhodamine coupled to an amino group; or a sulfonylchloride of Texas red coupled to a sulphydryl group. During the synthesis of these labeled components, conjugated oligonucleotides or PNAs can be purified, if desired, e.g., by high pressure liquid chromatography or other methods.

In general, synthetic methods for making oligonucleotides and PNAs (including labeled oligos and PNAs) is well known. For example, oligonucleotides can be synthesized chemically according to the solid phase phosphoramidite triester method described by Beaucage and Caruthers (1981), Tetrahedron Letts., 22(20):1859-1862, e.g., using a commercially available automated synthesizer, e.g., as described in Needham-VanDevanter et al. (1984) Nucleic Acids Res., 12:6159-6168. Synthesis of PNAs and modified oligonucleotides (e.g., oligonucleotides comprising 2′-O-methyl nucleotides and/or phosphorothioate, methylphosphonate, or boranophosphate linkages) are described in e.g., Oligonucleotides and Analogs (1991), IRL Press, New York; Shaw et al. (1993), Methods Mol. Biol.20:225-243; Nielsen et al. (1991), Science 254:1497-1500; and Shaw et al. (2000) Methods Enzymol. 313:226-257.

Oligonucleotides, including modified oligonucleotides (e.g., oligonucleotides comprising fluorophores and quenchers, 2′-O-methyl nucleotides, and/or phosphorothioate, methylphosphonate, or boranophosphate linkages) can also be ordered from a variety of commercial sources known to persons of skill. There are many commercial providers of oligo synthesis services, and thus, this is a broadly accessible technology. Any nucleic acid can be custom ordered from any of a variety of commercial sources, such as The Midland Certified Reagent Company (mcrc@oligos.com), The Great American Gene Company (www.genco.com), ExpressGen Inc. (www.expressgen.com), Operon Technologies Inc. (Alameda, Calif.) and many others. PNAs can be custom ordered from any of a variety of sources, such as PeptidoGenic (pkim@ccnet.com), HTI Bio-products, Inc. (www.htibio.com), BMA Biomedicals Ltd (U.K.), Bio-Synthesis, Inc., and many others. A variety of commercial suppliers produce standard and custom molecular beacons, including Cruachem (cruachem.com), Oswel Research Products Ltd. (UK; oswel.com), Research Genetics (a division of Invitrogen, Huntsville Ala. (resgen.com)), the Midland Certified Reagent Company (Midland, Tex. mcrc.com) and Gorilla Genomics, LLC (Alameda, Calif.).

Further details regarding methods of MB manufacture and use are found, e.g., in Leone et al. (1995) “Molecular beacon probes combined with amplification by NASBA enable homogenous real-time detection of RNA.” Nucleic Acids Res. 26:2150-2155; Tyagi and Kramer (1996) “Molecular beacons: probes that fluoresce upon hybridization” Nature Biotechnology 14:303-308; Blok and Kramer (1997) “Amplifiable hybridization probes containing a molecular switch” Mol Cell Probes 11:187-194; Hsuih et al. (1997) “Novel, ligation-dependent PCR assay for detection of hepatitis C in serum” J Clin Microbiol 34:501-507; Kostrikis et al. (1998) “Molecular beacons: spectral genotyping of human alleles” Science 279:1228-1229; Sokol et al. (1998) “Real time detection of DNA:RNA hybridization in living cells” Proc. Natl. Acad. Sci. U.S.A. 95:11538-11543; Tyagi et al. (1998) “Multicolor molecular beacons for allele discrimination” Nature Biotechnology 16:49-53; Bonnet et al. (1999) “Thermodynamic basis of the chemical specificity of structured DNA probes” Proc. Natl. Acad. Sci. U.S.A. 96:6171-6176; Fang et al. (1999) “Designing a novel molecular beacon for surface-immobilized DNA hybridization studies” J. Am. Chem. Soc. 121:2921-2922; Marras et al. (1999) “Multiplex detection of single-nucleotide variation using molecular beacons” Genet. Anal. Biomol. Eng. 14:151-156; and Vet et al. (1999) “Multiplex detection of four pathogenic retroviruses using molecular beacons” Proc. Natl. Acad. Sci. U.S.A. 96:6394-6399. Additional details regarding MB construction and use are found in the patent literature, e.g., U.S. Pat. No. 5,925,517 (Jul. 20, 1999) to Tyagi et al. entitled “Detectably labeled dual conformation oligonucleotide probes, assays and kits;” U.S. Pat. No. 6,150,097 to Tyagi et al (Nov. 21, 2000) entitled “Nucleic acid detection probes having non-FRET fluorescence quenching and kits and assays including such probes” and U.S. Pat. No. 6,037,130 to Tyagi et al (Mar. 14, 2000), entitled “Wavelength-shifting probes and primers and their use in assays and kits.”

PCR

Details regarding various PCR methods, including, e.g., asymmetric PCR, reverse transcription-PCR (rt-PCR), in situ PCR, quantitative PCR, real time PCR, and multiplex PCR, are well described in the literature. Details regarding PCR methods and applications thereof are found, e.g., in Sambrook et al., Molecular Cloning—A Laboratory Manual (3rd Ed.), Vol. 1-3, Cold Spring Harbor Laboratory, Cold Spring Harbor, N.Y. (2000); F. M. Ausubel et al. (eds.), Current Protocols in Molecular Biology, Current Protocols, a joint venture between Greene Publishing Associates, Inc. and John Wiley & Sons, Inc., (supplemented through 2002); Innis et al. (eds.), PCR Protocols: A Guide to Methods and Applications, Academic Press Inc., San Diego, Calif. (1990); J. P. V. Heuvel, PCR Protocols in Molecular Toxicology, CRC Press (1997); H. G. and A. Griffin, PCR Technology: Current Innovations, CRC Press (1994); Bagasra et al., (1997) In Situ PCR Techniques, Jossey-Bass; Bustin (2000) “Absolute quantification of mRNA using real-time reverse transcription polymerase chain reaction assays” Journal of Molecular Endocrinology 25:169-193; and Mackay et al. (2002) “Real-time PCR in virology” Nucleic Acids Res. 30:1292-1305, and references therein, among many other references. Additional details regarding PCR methods, including asymmetric PCR methods, are found in the patent literature, e.g., U.S. Pat. No. 6,391,544 (May 21, 2002) to Salituro et al. entitled “Method for using unequal primer concentrations for generating nucleic acid amplification products”; U.S. Pat. No. 5,066,584 (Nov. 19, 1991) to Gyllensten et al. entitled “Methods for generating single stranded DNA by the polymerase chain reaction”; and U.S. Pat. No. 5,691,146 (Nov. 25, 1997) to Mayrand entitled “Methods for combined PCR amplification and hybridization probing using doubly labeled fluorescent probes.”

In brief, PCR uses a pair of primers (typically synthetic oligonucleotides), each of which hybridizes to a strand of a double-stranded nucleic acid target that is amplified (the original template may be either single-stranded or double-stranded). The two primers typically flank the target region that is amplified. Template-dependent extension of the primers is catalyzed by a DNA polymerase, in the presence of deoxyribonucleoside triphosphates (typically dATP, dCTP, dGTP, and dTTP, although these can be replaced and/or supplemented with other dNTPs, e.g., a dNTP comprising a base analog that Watson-Crick base pairs like one of the conventional bases, e.g., uracil, inosine, or 7-deazaguanine), an aqueous buffer, and appropriate salts and metal cations (e.g., Mg²⁺). The PCR process involves cycles of three steps: denaturation (e.g., of double-stranded template and/or extension product), annealing (e.g., of one or both primers to template), and extension (e.g., of one or both primers to form double-stranded extension products). The cycles are typically thermal cycles; for example, cycles of denaturation at temperatures greater than about 90° C., annealing at 50-75° C., and extension at 72-78° C. A thermostable enzyme is thus preferred. Automated thermal cyclers, including integrated systems for real time detection of product, are commercially available, e.g., the ABI Prism® 7700 sequence detection system from Applied Biosystems (www.appliedbiosystems.com), or the iCycler iQ® real-time PCR detection system from Bio-Rad (www.biorad.com). Thermostable enzymes (including enzymes substantially lacking 5′ to 3′ nuclease activity), appropriate buffers, etc. are also widely commercially available, e.g., from Clontech (www.clontech.com), Invitrogen (www.invitrogen.com), Sigma-Aldrich (www.sigma-aldrich.com), and New England Biolabs (www.neb.com).

In symmetric PCR, the two primers are provided at equal concentrations, resulting in exponential amplification of the two strands of the target. In asymmetric PCR, one primer (e.g., the sense primer) is provided a higher concentration than the other (e.g., the antisense primer), resulting in the synthesis of more of one of the two complementary DNA strands (e.g., more of the strand into which the sense primer is incorporated). As mentioned previously, this can enhance detection of product, e.g., if the excess single strand is one to which a MB is complementary. In in situ PCR, PCR amplification is performed in fixed cells, and the amplified target can remain largely within the cell (or organelle etc.) which originally contained the nucleic acid template. Quantitative PCR can be employed, e.g., to determine the amount (relative or absolute) of target initially present in a sample. In real time PCR, product formation is monitored in real time. In real time quantitative PCR with fluorescent detection of product, a fluorescence threshold above background is typically assigned, and the time point at which each reaction's amplification plot reaches that threshold (defined as the threshold cycle number or Ct) is determined. The Ct value can be used to calculate the quantity of template initially present in each reaction. (Under a standard set of conditions, a lower or higher starting template concentration produces a higher or lower, respectively, Ct value.) In multiplex PCR, multiple target sequences can be amplified, detected, and/or quantitated simultaneously in one reaction mixture. In rt-PCR, reverse transcription of an RNA (e.g., an mRNA) produces a single-stranded DNA template that is used in subsequent PCR cycles. Combinations of such techniques (e.g., quantitative real time rt-PCR) are routine.

### EXAMPLES

The following sets forth a series of experiments that demonstrated the use of MBs in asymmetric PCR using nuclease-free DNA polymerases. It is understood that the examples and embodiments described herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

### Example 1

### Use of Nuclease-Free Taq with Asymmetric PCR Enhances the Performance of Molecular Beacons in Real Time PCR Assays-β-Actin Target

PCR primers to the human β-actin cDNA were synthesized using the standard phosphoramidite chemistry. The primers were desalted, concentrations were determined using UV spectrophotometry and the concentrations normalized. Molecular beacons to the human β-actin cDNA were synthesized using standard phosphoramidite chemistry. The molecular beacons were dual-labeled on synthesizer with 5′-FAM (6-carboxy-fluorescein) and 3′-Dabcyl. The molecular beacons were purified using denaturing ion pair reverse phase HPLC and the concentration determined using UV spectrophotometry. Double-stranded synthetic DNA templates to the human β-actin cDNA were prepared by PCR. The synthetic templates were purified using a Qiaquick PCR Purification Kit (Qiagen) and then quantified using a fluorometric Pico Green Assay (Molecular Probes). Hot start nuclease-free Taq DNA polymerase (i.e., Titanium ® Taq), 10×Taq buffer, and 10×nucleotide mix was purchased from Clontech (www.clontech.com). Reaction mixes for real time PCR experiments were assembled as follows: 1×PCR buffer; 2 mM MgCl₂; 1×nuclease-free Taq DNA polymerase; 2.5×10\hat{ }8, 2.5×10\hat{ }6, 2.5×10\hat{ }4, 2.5×10\hat{ }2 or 0 copies β-actin template; 400 nM sense primer; 444, 400, 364, 333, 308, 286, 267, 250, 235, 222, 210, and 200 nM antisense primer; Molecular Biology Grade H₂0 to a final reaction volume of 50 ul; and molecular beacon at 500 nM.

The reaction mixes were transferred to a 96 well optical PCR plate (Bio-Rad Laboratories), the plate was sealed using optical tape (Bio-Rad Laboratories), and then centrifuged. Real time PCR experiments were performed using an iCycler iQ real time PCR detection system. The cycling parameters included a single cycle at 95° C. for one minute to activate the nuclease-free Taq DNA polymerase, followed by 45 cycles of 95° C. for 30 seconds (denaturation step), 50° C. for 30 seconds (annealing step), and 72° C. for 30 seconds (extension step). Fluorescence (i.e., target amplification) was monitored in the FAM channel during the annealing step (i.e., at 50° C.) of each of the 45 cycles. The relative fluorescence data was baseline subtracted and plotted as a function of cycle number. Primer asymmetry rations were calculated as the [sense primer]/[antisense primer], where the sense primer primes the synthesis of and is incorporated into the strand of the target that is complementary to the target-recognition sequence (the loop) of the MB.

Specific molar ratios of sense-to-antisense primers were used to amplify the β-actin nucleic acid target, at four different concentrations of the target template, at a fixed concentration of beacon. At a molar ratio of sense-to-antisense primer of 0.9 (this asymmetric amplification favors the formation of the strand to which the beacon is identical, rather than the strand to which it is complementary), a regular or predictable transition temperature which permits the creation of a standard curve for interpretation of the data and quantitation of gene expression was not observed. In addition, the overall signal was low, ranging from 105-230 RFU (relative fluorescence units). When the molar concentrations of sense and antisense primers were equal (=symmetric amplification), the transition threshold appeared more regular and predictable over the template concentrations tested, and the overall fluorescent intensity range increased (119-233 RFU). Increasing the ratio of the sense primer to the antisense primer favors the formation of “free” target DNA (the strand to which the MB binds). As the molar ratio of sense-to-antisense increased from 1.1 to 2.0 we saw an increase in maximum (plateau) fluorescence from 271 to 675 RFU. Moreover, the absence of any collapse in the RT-PCR curves at the highest ratio tested indicated that even higher ratios can provide further improvement on the method.

At each of four different template concentrations tested, the increase in fluorescence with increasing sense-to-antisense asymmetry was notable, but in addition it was apparent that with increasing asymmetry the transition threshold was shifted to the left, i.e. at a lower PCR cycle number. This is a demonstration that the sensitivity of the assay is improved with increasing asymmetry, at all target concentrations tested.

### Example 2

### Use of Nuclease-Free Taq with Asymmetric PCR Enhances the Performance of Molecular Beacons in Real Time PCR Assays—Multiple Targets

PCR primers (sense and antisense) to eight human cDNAs (targets F6, E2, E5, A2, B 1, A5, B2, and A6) were synthesized using the standard phosphoramidite chemistry. The primers were desalted, concentrations were determined using UV spectrophotometry and the concentrations were normalized. Molecular beacons (one to each of the eight cDNAs) were synthesized using standard phosphoramidite chemistry. The molecular beacons were dual-labeled on synthesizer with 5 ′-FAM and 3 ′-Dabcyl. The molecular beacons were purified using denaturing ion pair reverse phase HPLC and the concentration determined using UV spectrophotometry. Double-stranded synthetic DNA templates to the eight target cDNAs were prepared by PCR. The synthetic templates were purified using Qiaquick PCR Purification Kit (Qiagen) and then quantified using a fluorometric Pico Green Assay (Molecular Probes). Hot start nuclease free Taq DNA polymerase (i.e., Titanium® Taq) was purchased from Clontech (www.clontech.com). Reaction mixes for real time PCR experiments were assembled as follows: 20 mM TrisHCl, pH 8.0, 3 mM MgCl2, 50 mM KCl, 200 uM dNTPs, 0.4 units nuclease free Taq DNA polymerase; 10\hat{ }7 copies double stranded DNA template; and either 600 nM sense primer and 600 nM antisense primer, 200 nM sense and 200 nM antisense, or 600 nM sense primer and 200 nM antisense; molecular beacon at 100 nM; and Molecular Biology Grade H2O (Sigma) to a final reaction volume of 50 ul. ROX reference dye was added to normalize the fluorescent signal as recommended by the supplier (Invitrogen).

The reaction mixes were set up in a 96 well optical PCR plate (Applied Biosystems), the plate was sealed using optical tape (Applied Biosystems), and then centrifuged. Real time PCR experiments were performed using the ABI 7700 Sequence Detector. The cycling parameters included a single cycle at 95° C. for 3 minutes to activate the nuclease-free Taq DNA polymerase, followed by 45 cycles of 95° C. for 30 seconds (denaturation step), 55° C. for 30 seconds (annealing step), and 72° C. for 30 seconds (extension step). Fluorescence (i.e., target amplification) was monitored in the FAM channel during the annealing step (i.e., at 55° C.) of each of the 45 cycles. Using the ABI software, the baseline was set at cycles 5-15. The relative fluorescence (ΔRn) data was baseline subtracted and plotted as a function of cycle number. The cycle threshold was selected above the noise within the exponential phase of the amplification plot. Primer asymmetry ratios were calculated as the [sense primer]/[antisense primer].

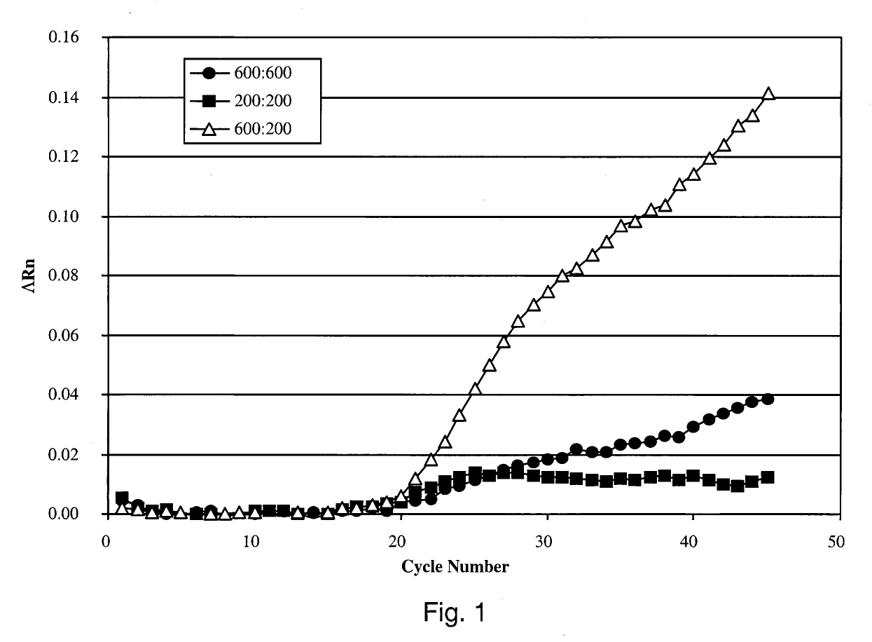

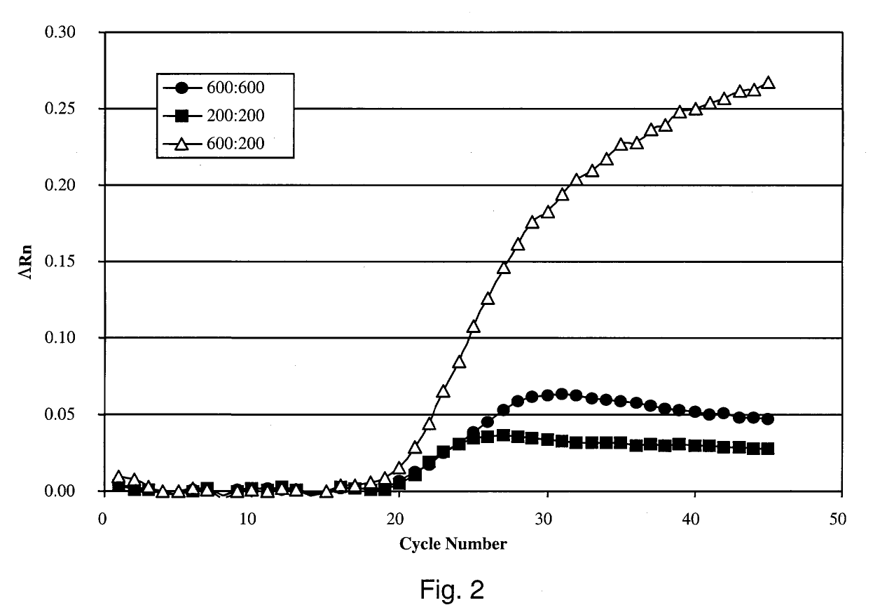

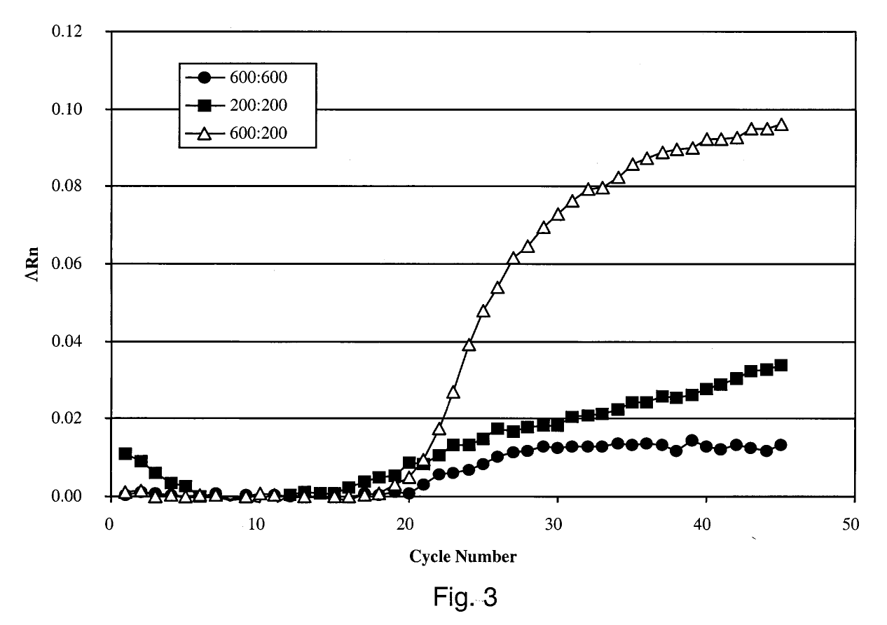

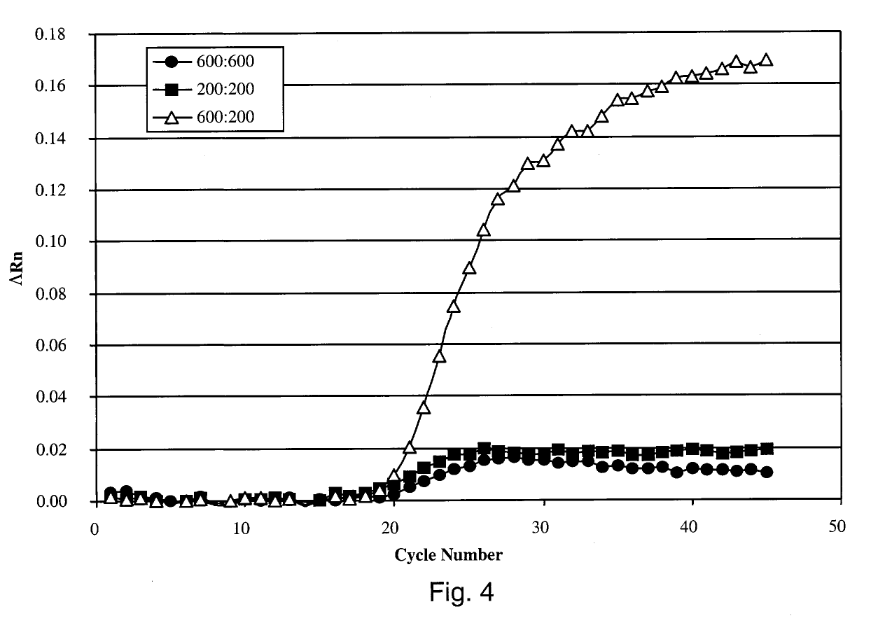

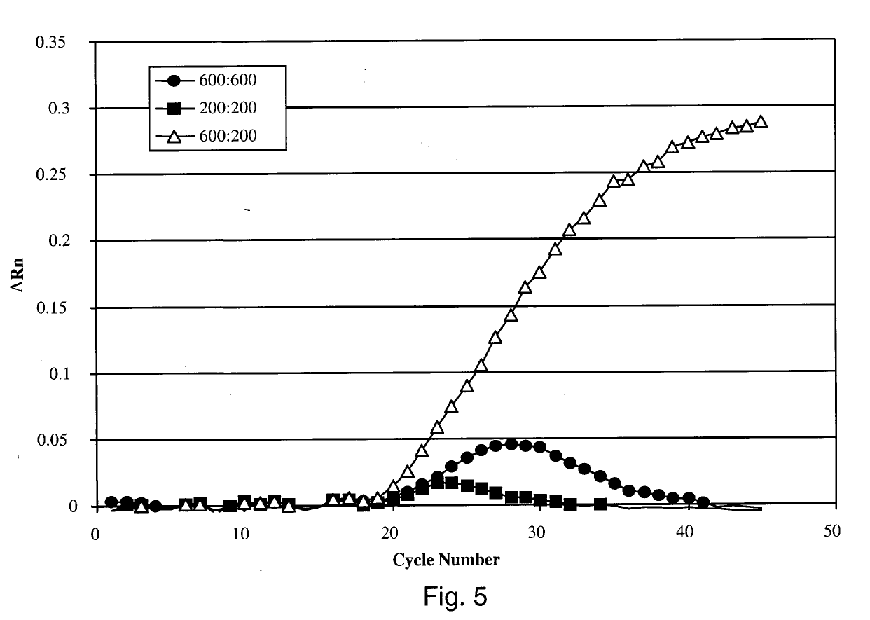

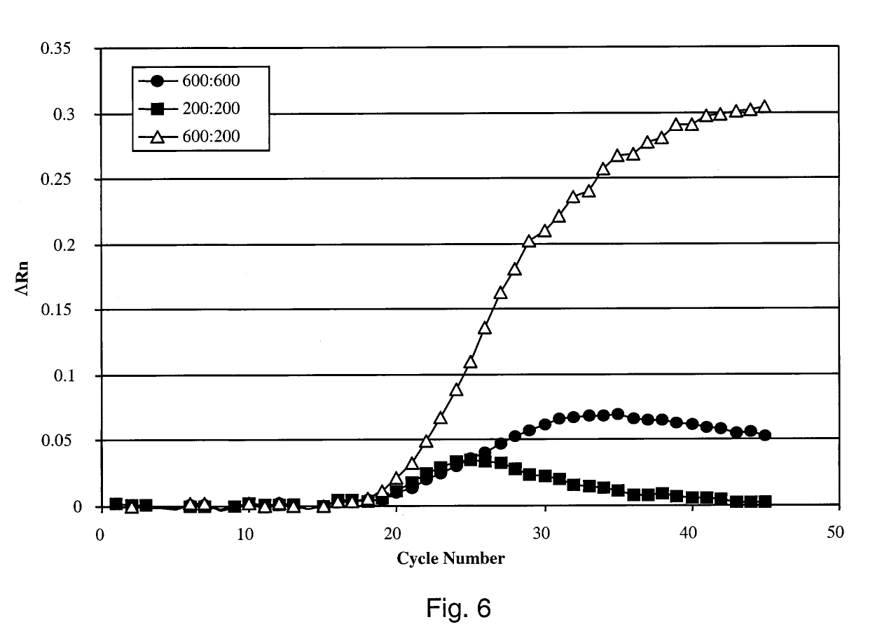

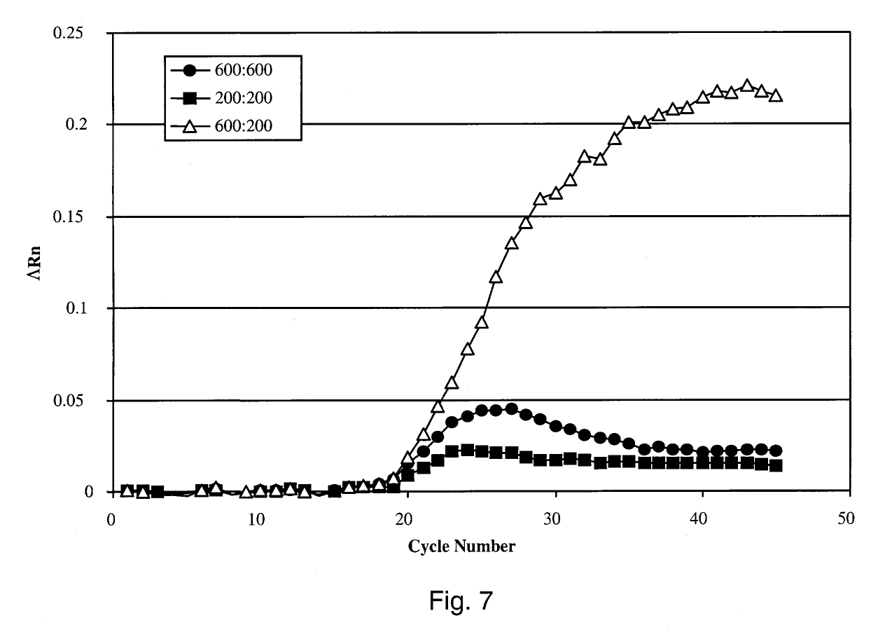

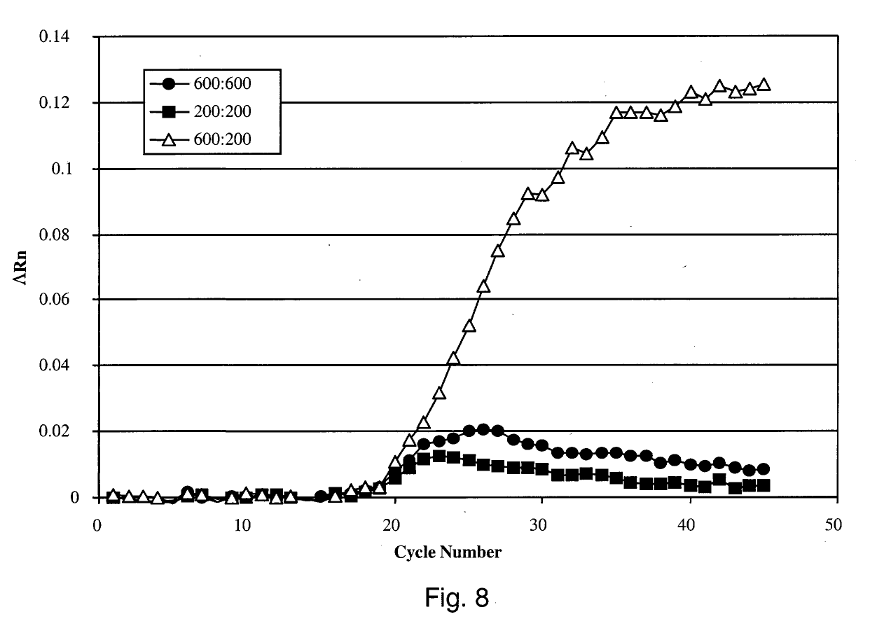

Results are illustrated in Tables 1 and 2 and in FIGS. 1-8, which present the amplification plots (i.e., the relative fluorescence intensity measured at each cycle plotted versus cycle number) for the eight cDNAs undergoing symmetric amplification (with either 200 nM, squares, or 600 nM, circles, of the relevant sense and antisense primers) or asymmetric amplification (with 600 nM sense:200 nM antisense primer, triangles). For each template tested, the asymmetric amplification increased the sensitivity of the assay (e.g., increased the maximum fluorescence observed and/or shifted the Ct to a lower cycle number) and provided a wider dynamic linear signal range.

TABLE 1
Ct Values at Threshold 0.01, Baseline 5-15

| | 600:600 | 200:200 | 600:200 |
|---|---|---|---|
| target F6 | 24.32 | 22.91 | 20.78 |
| target E2 | 20.59 | 20.90 | 19.20 |
| target E5 | 25.87 | 19.36 | 21.05 |
| target A2 | 23.20 | 21.28 | 20.06 |
| target B1 | 20.98 | 21.55 | 19.47 |
| target A5 | 19.91 | 19.85 | 18.86 |
| target B2 | 19.40 | 20.25 | 19.27 |
| target A6 | 20.72 | 21.42 | 19.90 |

TABLE 2
ΔRn Max Values (at cycle 45)

| | 600:600 | 200:200 | 600:200 |
|---|---|---|---|
| target F6 | 0.038 | 0.012 | 0.141 |
| target E2 | 0.047 | 0.028 | 0.267 |
| target E5 | 0.013 | 0.034 | 0.096 |
| target A2 | 0.010 | 0.019 | 0.169 |
| target B1 | −0.003 | −0.004 | 0.288 |
| target A5 | 0.053 | 0.003 | 0.303 |
| target B2 | 0.021 | 0.014 | 0.215 |
| target A6 | 0.008 | 0.004 | 0.125 |

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method for performing combined amplification and detection of a nucleic acid target, the method comprising:
providing a molecular beacon comprising a region of complementarity to a first region of a first strand of the nucleic acid target;
providing a first primer comprising a region of identity with a second region of the first strand of the nucleic acid target;
providing a second primer comprising a region of complementarity to a third region of the first strand of the nucleic acid target, the third region being 3′ of the first region, and the first region being 3′ of the second region;
wherein the first primer is provided at a concentration that is at least about 1.3 times that of the second primer;
providing a template nucleic acid comprising the first strand of the nucleic acid target, a second strand of the nucleic acid target that is complementary to the first strand, or both;
providing a polymerase substantially lacking 5′ to 3′ nuclease activity;
amplifying the target nucleic acid by subjecting the template nucleic acid, the first and second primers, the molecular beacon, and the polymerase to cycles comprising denaturation, annealing, and extension steps; and,
detecting a signal from the molecular beacon at at least one time point during or after the cycles.

2. The method of claim 1, wherein the first primer is provided at a concentration that is at least about two times the concentration of the second primer.

3. The method of claim 1, wherein the first primer is provided at a concentration that is at least about three times the concentration of the second primer.

4. The method of claim 1, wherein the cycles of denaturation, annealing, and extension steps comprise thermal cycles.

5. The method of claim 4, wherein the polymerase is a thermostable polymerase.

6. The method of claim 5, wherein the thermostable polymerase is a DNA polymerase, or a modified form thereof, from a Thermus species.

7. The method of claim 1, wherein the polymerase has a 5′ to 3′ nuclease activity that is ten percent or less than that of the Thermus aquaticus Taq DNA polymerase under typical reaction conditions.

8. The method of claim 1, wherein the polymerase has a 5′ to 3′ nuclease activity that is five percent or less than that of the Thermus aquaticus Taq DNA polymerase under typical reaction conditions.

9. The method of claim 1, wherein the polymerase has no detectable 5′ to 3′ nuclease activity under typical reaction conditions.

10. The method of claim 1, wherein the signal from the molecular beacon is detected during the annealing step of each cycle.

11. The method of claim 1, wherein detecting a signal from the molecular beacon comprises detecting a fluorescent emission from the molecular beacon.

12. The method of claim 11, wherein detecting the fluorescent emission comprises measuring the intensity of the fluorescent emission.

13. The method of claim 1, wherein the template nucleic acid is a single-stranded DNA product of a reverse transcription reaction.

14. The method of claim 1, wherein the template nucleic acid is located within one or more fixed cells.

15. The method of claim 1, wherein the template nucleic acid is bound to a matrix.

16. The method of claim 1, wherein:
providing a molecular beacon comprises providing two or more molecular beacons, each of which comprises a region of complementarity to a strand of a different nucleic acid target;
providing a first and second primer comprises providing a first and second primer for each different nucleic acid target, wherein each first primer is provided at a concentration that is at least about 1.3 times that of the corresponding second primer;
providing a template nucleic acid comprises providing a template nucleic acid for each different nucleic acid target;
amplifying the target nucleic acid comprises amplifying each target nucleic acid; and,
detecting a signal from the molecular beacon comprises detecting a signal from each of the two or more molecular beacons.

17. The method of claim 16, wherein the template nucleic acids form an array on a matrix, in which array each template nucleic acid is bound to the matrix at a unique location.

18. The method of claim 16, wherein the method is used for single nucleotide discrimination.

19. A method for performing combined amplification and detection of a nucleic acid target, the method comprising:
providing a molecular beacon comprising a region of complementarity to a first region of a first strand of the nucleic acid target, the molecular beacon being resistant to 5′ to 3′ nuclease activity;
providing a first primer comprising a region of identity with a second region of the first strand of the nucleic acid target;
providing a second primer comprising a region of complementarity to a third region of the first strand of the nucleic acid target, the third region being 3′ of the first region, and the first region being 3′ of the second region;
wherein the first primer is provided at a concentration that is at least about 1.3 times that of the second primer;
providing a template nucleic acid comprising the first strand of the nucleic acid target, a second strand of the nucleic acid target that is complementary to the first strand, or both;
providing a polymerase;
amplifying the target nucleic acid by subjecting the template nucleic acid, the first and second primers, the molecular beacon, and the polymerase to cycles comprising denaturation, annealing, and extension steps; and,
detecting a signal from the molecular beacon at at least one time point during or after the cycles.

20. The method of claim 19, wherein the first primer is provided at a concentration that is at least about two times the concentration of the second primer.

21. The method of claim 19, wherein the first primer is provided at a concentration that is at least about three times the concentration of the second primer.

22. The method of claim 19, wherein the cycles of denaturation, annealing, and extension steps comprise thermal cycles.

23. The method of claim 22, wherein the polymerase is a thermostable polymerase.

24. The method of claim 19, wherein the signal from the molecular beacon is detected during the annealing step of each cycle.

25. The method of claim 19, wherein detecting a signal from the molecular beacon comprises detecting a fluorescent emission from the molecular beacon.

26. The method of claim 25, wherein detecting a fluorescent emission comprises measuring the intensity of the fluorescent emission.

27. The method of claim 19, wherein the template nucleic acid is a single-stranded DNA product of a reverse transcription reaction.

28. The method of claim 19, wherein the template nucleic acid is located within one or more fixed cells.

29. The method of claim 19, wherein the template nucleic acid is bound to a matrix.

30. The method of claim 19, wherein the molecular beacon comprises a peptide nucleic acid.

31. The method of claim 19, wherein the molecular beacon comprises one or more 2′-O-methyl nucleotides.

32. The method of claim 19, wherein the molecular beacon comprises one or more phosphorothioate linkages.

33. The method of claim 19, wherein:
providing a molecular beacon comprises providing two or more molecular beacons, each of which comprises a region of complementarity to a strand of a different nucleic acid target, and each of which is resistant to 5′ to 3′ nuclease activity;
providing a first and second primer comprises providing a first and second primer for each different nucleic acid target, wherein each first primer is provided at a concentration that is at least about 1.3 times that of the corresponding second primer;
providing a template nucleic acid comprises providing a template nucleic acid for each different nucleic acid target;
amplifying the target nucleic acid comprises amplifying each target nucleic acid; and,
detecting a signal from the molecular beacon comprises detecting a signal from each of the two or more molecular beacons.

34. The method of claim 33, wherein the template nucleic acids form an array on a matrix, in which array each template nucleic acid is bound to the matrix at a unique location.

35. The method of claim 19, wherein the method is used for single nucleotide discrimination.

36. A composition comprising:
a molecular beacon comprising a region of complementarity to a first region of a first strand of a nucleic acid target;
a first primer comprising a region of identity with a second region of the first strand of the nucleic acid target;
a second primer comprising a region of complementarity to a third region of the first strand of the nucleic acid target, the third region being 3′ of the first region, and the first region being 3′ of the second region, wherein the first primer is present at a concentration that is at least about 1.3 times that of the second primer; and,
a polymerase substantially lacking 5′ to 3′ nuclease activity.

37. The composition of claim 36, wherein the first primer is present at a concentration that is at least about two times the concentration of the second primer.

38. The composition of claim 36, wherein the first primer is present at a concentration that is at least about three times the concentration of the second primer.

39. The composition of claim 36, further comprising a template nucleic acid, the template comprising the first strand of the nucleic acid target, a second strand of the nucleic acid target that is complementary to the first strand, or both.

40. The composition of claim 36, wherein the polymerase is a thermostable polymerase.

41. The composition of claim 40, wherein the thermostable polymerase is a DNA polymerase, or a modified form thereof, from a Thermus species.

42. The composition of claim 36, wherein the polymerase has a 5′ to 3′ nuclease activity that is ten percent or less than that of the Thermus aquaticus Taq DNA polymerase under typical reaction conditions.

43. The composition of claim 36, wherein the polymerase has a 5′ to 3′ nuclease activity that is five percent or less than that of the Thermus aquaticus Taq DNA polymerase under typical reaction conditions.

44. The composition of claim 36, wherein the polymerase has no detectable 5′ to 3′ nuclease activity under typical reaction conditions.

45. The composition of claim 36, wherein the composition is formed at one or more positions on an array.

46. The composition of claim 36, wherein the composition is contained in a thermal cycler.

47. A kit comprising each of the components of the composition of claim 36 packaged in one or more containers.

48. The kit of claim 47, further comprising one or more of: a buffer, a standard target for calibrating a detection reaction, instructions for using the components to detect and/or quantitate the nucleic acid target, or packaging materials.

49. A composition comprising:
a molecular beacon comprising a region of complementarity to a first region of a first strand of a nucleic acid target, the molecular beacon being resistant to 5′ to 3′ nuclease activity;
a first primer comprising a region of identity with a second region of the first strand of the nucleic acid target; and,
a second primer comprising a region of complementarity to a third region of the first strand of the nucleic acid target, the third region being 3′ of the first region, and the first region being 3′ of the second region;
wherein the first primer is present at a concentration that is at least about 1.3 times that of the second primer.

50. The composition of claim 49, wherein the first primer is present at a concentration that is at least about two times the concentration of the second primer.

51. The composition of claim 49, wherein the first primer is present at a concentration that is at least about three times the concentration of the second primer.

52. The composition of claim 49, wherein the molecular beacon comprises a peptide nucleic acid.

53. The composition of claim 49, wherein the molecular beacon comprises one or more 2′-O-methyl nucleotides.

54. The composition of claim 49, wherein the molecular beacon comprises one or more phosphorothioate linkages.

55. The composition of claim 49, further comprising a template nucleic acid, the template comprising the first strand of the nucleic acid target, a second strand of the nucleic acid target that is complementary to the first strand, or both.

56. The composition of claim 49, further comprising a polymerase.

57. The composition of claim 56, wherein the polymerase is a thermostable polymerase.

58. The composition of claim 49, wherein the composition is formed at one or more positions on an array.

59. The composition of claim 49, wherein the composition is contained in a thermal cycler.

60. A kit comprising each of the components of the composition of claim 49 packaged in one or more containers.

61. The kit of claim 60, further comprising one or more of: a buffer, a standard target for calibrating a detection reaction, instructions for using the components to detect and/or quantitate the nucleic acid target, or packaging materials.
